(19) United States
(12) Patent Application Publication
(10) Pub. No.: US 2016/0206704 A1
(43) Pub. Date: Jul. 21, 2016

(54) METHOD

(71) Applicant: Isis Innovation Limited, Oxford (GB)

(72) Inventor: Robert MacLaren, Oxford (GB)

(21) Appl. No.: 14/598,948

(22) Filed: Jan. 16, 2015

(51) Int. Cl. A61K 38/45; A61K 48/00; C12N 7/00; A61F 9/00

(52) U.S. Cl. CPC A61K 38/45; A61F 9/0017; A61K 48/0075; A61K 48/0058; C12N 7/00; C12Y 205/0106; C12N 2750/14143

(57) ABSTRACT

A method of using a vector comprising a transgene to treat a disease or condition of the eye, the method comprising the steps: (a) administering a solution to a mammalian subject by subretinal injection in an amount effective to at least partially detach the retina to form a subretinal bleb, wherein the solution does not comprise the vector; and (b) administering a medicament composition by subretinal injection into the bleb formed by step (a), wherein the medicament comprises the vector and is injected in an amount effective to treat the disease or condition; wherein the transgene is expressible in cells of the mammalian subject.

### FIELD OF THE INVENTION

The present invention relates to methods of delivering medicaments to the eye. More specifically, the present invention relates to improved methods for delivering medicaments to cells at the back of the eye, such as cells of the neurosensory retina, retinal pigment epithelium or choroid.

### BACKGROUND TO THE INVENTION

Treatment of eye diseases by the delivery of medicaments to the eye is becoming increasingly viable. In particular, there have been a number of significant recent advances in gene therapy of eye diseases. For example, AAV vectors have been investigated for the treatment of Leber congenital amaurosis (Cideciyan, A. V. et al. (2013) Proc. Natl. Acad. Sci. USA 110: E517-25).

The treatment of eye diseases may require medicaments (e.g. viral vector particles in the case of gene therapy) to be delivered to cells at the back of the eye. For example, cells of the neurosensory retina, retinal pigment epithelium and choroid are amongst those that it may be desirable to target by medicaments such as gene therapy vectors. A number of techniques are known for the delivery of such medicaments, including direct retinal, subretinal and intravitreal injection.

Subretinal injection, during which localised detachment of the retina occurs, is a preferred method for the delivery of medicaments to the back of the eye. Detachment of the retina aids the delivery of the medicaments (e.g. gene therapy vectors) to the subretinal space in order to target certain cells appropriately, for example photoreceptor and retinal pigment epithelial cells.

The amount of detachment of the retina during subretinal injection may be unpredictable. This issue is particularly acute when the patient has certain diseases, such as end-stage choroideremia, where the variable nature of the retinal degeneration associated with the disease renders it likely that different volumes of subretinal fluid will be required to detach the residual retina from the underlying choroid. This may result in variable doses of medicament being administered while eliciting the detachment.

Moreover, unpredictable retinal detachment may risk damage to the fovea. Because the human fovea is a delicate structure which is responsible for central high resolution vision, it is vitally important to avoid damage to this structure during the procedure of delivering the medicament.

Furthermore, any unexpected retinal tears or breaks occurring during the injection could result in escape of the medicament into the vitreous. This escape may also cause problems with delivering an accurate dose of the medicament and/or give rise to adverse off-target effects.

### SUMMARY OF THE INVENTION

The present inventors have developed an improved subretinal injection method to address these problems. During the improved method, the retinal detachment procedure is completely separated from the process of administering the medicament.

Specifically, the present inventors have surprisingly found that medicaments can be delivered more accurately and safely by using a two-step method in which a localised retinal detachment is created by the subretinal injection of a first solution. The first solution does not comprise the medicament. A second subretinal injection is then used to deliver the medicament into the subretinal fluid of the bleb created by the first subretinal injection. Because the injection delivering the medicament is not being used to detach the retina, a specific volume of solution may be injected in this second step. Accordingly, the improved method enables delivery of more accurate doses.

The improved method has the additional benefit that the more unpredictable part of the procedure (i.e. the creation of the bleb) is performed before any vector is administered to the patient. This allows for the management of any surgical complications with standard techniques (e.g. laser and gas), without concerns about dissemination of the medicament more widely than the intended target. Moreover, because no vector would have been administered at that stage if a complication arises during the creation of the bleb, patients would be able to be left to recover and the procedure re-attempted at a later date.

Accordingly, in a first aspect, the present invention provides a method of using a vector comprising a transgene to treat a disease or condition of the eye, the method comprising the steps:
(a) administering a solution to a mammalian subject by subretinal injection in an amount effective to at least partially detach the retina to form a subretinal bleb, wherein the solution does not comprise the vector; and
(b) administering a medicament composition by subretinal injection into the bleb formed by step (a), wherein the medicament comprises the vector and is injected in an amount effective to treat the disease or condition;

wherein the transgene is expressible in cells of the mammalian subject.

In another aspect, the present invention provides a method of treatment of a disease or condition of the eye, the method comprising administering a vector comprising a transgene by the steps:
(a) administering a solution to a mammalian subject by subretinal injection in an amount effective to at least partially detach the retina to form a subretinal bleb, wherein the solution does not comprise the vector; and
(b) administering a medicament composition by subretinal injection into the bleb formed by step (a), wherein the medicament comprises the vector and is injected in an amount effective to treat the disease or condition;

wherein the transgene is expressible in cells of the mammalian subject.

The volume of solution injected in step (a) to at least partially detach the retina may be, for example, about 10-1000 μL, for example about 50-1000, 100-1000, 250-1000, 500-1000, 10-500, 50-500, 100-500, 250-500 μL. The volume may be, for example, about 10, 50, 100, 200, 300, 400, 500, 600, 700, 800, 900 or 1000 μL.

The volume of the medicament composition injected in step (b) may be, for example, about 10-500 μL, for example about 50-500, 100-500, 200-500, 300-500, 400-500, 50-250, 100-250 or 200-250 μL. The volume may be, for example, about 10, 50, 100, 150, 200, 250, 300, 350, 400, 450 or 500 μL. Preferably, the volume of the medicament composition injected in step (b) is 100 μL. Larger volumes may increase the risk of stretching the retina, while smaller volumes may be difficult to see.

During step (b), the medicament composition is injected into the subretinal fluid of the bleb formed by step (a).

In one embodiment, the vector is a viral vector, preferably in the form of a viral vector particle.

In another embodiment, the viral vector is an AAV, retroviral, lentiviral or adenoviral vector. The viral vector may be, for example, an AAV, retroviral, lentiviral or adenoviral vector particle.

The viral vector may, for example, be in a suspension at a concentration of about 1×10¹² genome particles per mL. A typical dose of viral vector of about 1×10¹¹ genome particles may, for example, be administered (i.e. about a 100 μL dose of viral vector at a concentration of about 1×10¹² genome particles per mL). The skilled person is readily able to adjust the dose, volume and concentration of the viral vector as necessary.

In another embodiment, the transgene is a gene encoding Rab escort protein-1 (REP1) or retinal pigment epithelium-specific 65 kDa protein (RPE65). The transgene may, for example, comprise a Rab escort protein-1 (REP1) or retinal pigment epithelium-specific 65 kDa protein (RPE65) open reading frame operably linked to an expression control sequence to promote expression in cells of the eye of the mammalian subject. The REP1 gene is sometimes known as the CHM gene.

In another embodiment, the vector is delivered to cells of the neurosensory retina (e.g. photoreceptor cells), retinal pigment epithelium and/or choroid.

In another embodiment, the medicament composition is administered by subretinal injection through the same retinotomy used to administer the solution (i.e. the solution of step (a) that does not comprise the vector). Preferably, the subretinal injections are carried out using a subretinal injection needle to create self-sealing entry points in the neurosensory retina.

In another embodiment, the area of the retina to be injected is dyed with a blue vital dye before the subretinal injection of step (a) is carried out.

In another embodiment, the subretinal injections are at positions greater than or equal to about 1 mm from the fovea, such as about 1.5, 2.0, 2.5, 3.0, 3.5, 4.0, 4.5, 5.0 mm from the fovea or further still.

In another embodiment, the subretinal injections of steps (a) and/or (b) are made in a series of successive pulses to stretch and blanch the retina.

In another embodiment, the solution injected in step (a) is balanced salt solution (BSS) or a similar buffer solution matched to the pH and osmolality of the subretinal space.

In another embodiment, the disease or condition is a retinal dystrophy.

In another embodiment, the disease or condition is choroideremia, Leber congenital amaurosis, cone-rod dystrophy, macular dystrophy, cone dystrophy, achromatopsia, retinitis pigmentosa or age-related macular degeneration.

In a preferred embodiment, the method is for treating choroideremia.

In another aspect, the present invention provides a method of delivering a medicament to the eye comprising the steps:
(a) administering a first solution by subretinal injection in an amount effective to at least partially detach the retina to form a subretinal bleb, wherein the first solution does not comprise the medicament; and
(b) administering a second solution by subretinal injection into the bleb formed by step (a), wherein the second solution comprises the medicament.

The volume of the first solution injected in step (a) to at least partially detach the retina may be, for example, about 10-1000 μL, for example about 50-1000, 100-1000, 250-1000, 500-1000, 10-500, 50-500, 100-500, 250-500 μL. The volume may be, for example, about 10, 50, 100, 200, 300, 400, 500, 600, 700, 800, 900 or 1000 μL.

The volume of the second solution injected in step (b) may be, for example, about 10-500 μL, for example about 50-500, 100-500, 200-500, 300-500, 400-500, 50-250, 100-250 or 200-250 μL. The volume may be, for example, about 10, 50, 100, 150, 200, 250, 300, 350, 400, 450 or 500 μL. Preferably, the volume of the second solution injected in step (b) is 100 μL. Larger volumes may increase the risk of stretching the retina, while smaller volumes may be difficult to see.

During step (b), the second solution is injected into the subretinal fluid of the bleb formed by step (a).

In one embodiment, the delivery of the medicament is to cells of the neurosensory retina (e.g. photoreceptor cells), retinal pigment epithelium and/or choroid.

In another embodiment, the subretinal injection of step (b) is made through the same retinotomy as the subretinal injection of step (a). Preferably, the subretinal injections are carried out using a subretinal injection needle to create self-sealing entry points in the neurosensory retina.

In another embodiment, the area of the retina to be injected is dyed with a blue vital dye before the subretinal injection of step (a) is carried out.

In another embodiment, the subretinal injections are at positions greater than or equal to about 1 mm from the fovea, such as about 1.5, 2.0, 2.5, 3.0, 3.5, 4.0, 4.5, 5.0 mm from the fovea or further still.

In another embodiment, the subretinal injections of steps (a) and/or (b) are made in a series of successive pulses to stretch and blanch the retina.

In a preferred embodiment, the medicament comprises a viral vector, preferably in the form of a viral vector particle, comprising a transgene. The viral vector may be, for example, an AAV, retroviral, lentiviral or adenoviral vector or vector particle.

In one embodiment, the transgene is a gene encoding Rab escort protein-1 (REP1) or retinal pigment epithelium-specific 65 kDa protein (RPE65). The REP1 gene is sometimes known as the CHM gene.

In another embodiment, the first solution is balanced salt solution (BSS) or a similar buffer solution matched to the pH and osmolality of the subretinal space.

In another aspect, the present invention provides a method of treating a disease comprising the method of the invention for delivering a medicament.

In one embodiment, the method is for treating a retinal dystrophy.

In another embodiment, the method is for treating choroideremia, Leber congenital amaurosis, cone-rod dystrophy, macular dystrophy, cone dystrophy, achromatopsia, retinitis pigmentosa or age-related macular degeneration.

In a preferred embodiment, the method is for treating choroideremia.

In another aspect, the present invention provides a kit comprising: (i) the medicament described herein (e.g. the vector); and (ii) a solution that does not comprise the medicament for use in at least partially detaching the retina of a mammalian subject to form a subretinal bleb. Preferably the solution of component (ii) is balanced salt solution (BSS) or a similar buffer solution matched to the pH and osmolality of the subretinal space.

The kit may also additionally comprise the “injector syringe” and/or modified syringe lock as described herein.

In a particularly preferred embodiment, the present invention provides a method of using an AAV vector comprising a gene encoding Rab escort protein-1 (REP1) to treat choroideremia, the method comprising the steps:
(a) administering a solution to a mammalian subject by subretinal injection in an amount effective to at least partially detach the retina to form a subretinal bleb, wherein the solution does not comprise the vector; and
(b) administering a medicament composition by subretinal injection into the bleb formed by step (a), wherein the medicament comprises the AAV vector comprising a gene encoding Rab escort protein-1 (REP1) and is injected in an amount effective to treat the choroideremia;

wherein the Rab escort protein-1 (REP1) is expressible in cells of the mammalian subject.

In another preferred embodiment, the present invention provides a method of treatment of choroideremia, the method comprising administering an AAV vector comprising a gene encoding Rab escort protein-1 (REP1) by the steps:
(a) administering a solution to a mammalian subject by subretinal injection in an amount effective to at least partially detach the retina to form a subretinal bleb, wherein the solution does not comprise the vector; and
(b) administering a medicament composition by subretinal injection into the bleb formed by step (a), wherein the medicament comprises the AAV vector comprising a gene encoding Rab escort protein-1 (REP1) and is injected in an amount effective to treat the choroideremia;

wherein the Rab escort protein-1 (REP1) is expressible in cells of the mammalian subject.

In another preferred embodiment, the present invention provides a method of delivering an AAV vector comprising a gene encoding Rab escort protein-1 (REP1) to the eye comprising the steps:
(a) administering a first solution by subretinal injection in an amount effective to at least partially detach the retina to form a subretinal bleb, wherein the first solution does not comprise the vector; and
(b) administering a second solution by subretinal injection into the bleb formed by step (a), wherein the second solution comprises the AAV vector comprising a gene encoding Rab escort protein-1 (REP1).

In another particularly preferred embodiment, the present invention provides a method of using an AAV vector comprising a gene encoding retinal pigment epithelium-specific 65 kDa protein (RPE65) to treat Leber congenital amaurosis, the method comprising the steps:
(a) administering a solution to a mammalian subject by subretinal injection in an amount effective to at least partially detach the retina to form a subretinal bleb, wherein the solution does not comprise the vector; and
(b) administering a medicament composition by subretinal injection into the bleb formed by step (a), wherein the medicament comprises the AAV vector comprising a gene encoding retinal pigment epithelium-specific 65 kDa protein (RPE65) and is injected in an amount effective to treat the Leber congenital amaurosis;

wherein the retinal pigment epithelium-specific 65 kDa protein (RPE65) is expressible in cells of the mammalian subject.

In another preferred embodiment, the present invention provides a method of treatment of Leber congenital amaurosis, the method comprising administering an AAV vector comprising a gene encoding retinal pigment epithelium-specific 65 kDa protein (RPE65) by the steps:
(a) administering a solution to a mammalian subject by subretinal injection in an amount effective to at least partially detach the retina to form a subretinal bleb, wherein the solution does not comprise the vector; and
(b) administering a medicament composition by subretinal injection into the bleb formed by step (a), wherein the medicament comprises the AAV vector comprising a gene encoding retinal pigment epithelium-specific 65 kDa protein (RPE65) and is injected in an amount effective to treat the Leber congenital amaurosis;

wherein the retinal pigment epithelium-specific 65 kDa protein (RPE65) is expressible in cells of the mammalian subject.

In another preferred embodiment, the present invention provides a method of delivering an AAV vector comprising a gene encoding retinal pigment epithelium-specific 65 kDa protein (RPE65) to the eye comprising the steps:
(a) administering a first solution by subretinal injection in an amount effective to at least partially detach the retina to form a subretinal bleb, wherein the first solution does not comprise the vector; and
(b) administering a second solution by subretinal injection into the bleb formed by step (a), wherein the second solution comprises the AAV vector comprising a gene encoding retinal pigment epithelium-specific 65 kDa protein (RPE65).

### DESCRIPTION OF THE DRAWINGS

FIG. 1A depicts an example of a “drawing-up syringe”. A 1 mL BD Luer-Lok syringe is fitted with a 19 G BD Microlance needle and is shown containing 0.30 mL of vector suspension.

FIG. 1B depicts an example of an “injector syringe”. The syringe is shown with its plunger removed and the rubber bung reinserted to the 0.35 mL mark ready for accepting the vector suspension through the open tip.

FIG. 1C depicts an example of an “injector syringe” fitted with a DORC 41 G Teflon subretinal injection needle.

FIG. 2A depicts a syringe lock from the Bausch & Lomb Viscous Fluid Pack (CX5710, Bausch & Lomb Inc, NY, USA; and FIG. 2B depicts the syringe lock that has been custom modified to fit an 8.0 mm rubber O-ring.

An “injector syringe” can be fitted onto the modified syringe lock depicted in FIG. 2B using an O-ring to provide an air-tight seal. The modified syringe lock can then be connected via a standard Luer-Lok air-filter to Easy Connection Tubing.

FIG. 3A depicts the “injector syringe” and modified syringe lock disconnected from the air filter and tubing. FIG. 3B depicts the “injector syringe”, modified syringe lock, air filter and tubing as connected.

### DETAILED DESCRIPTION OF THE INVENTION

Various preferred features and embodiments of the present invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F., and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J., and Kahn, A. (1996) DNA Isolation and Sequencing: Essential Techniques, John Wiley & Sons; Polak, J. M., and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M., and Dahlberg, J. E. (1992) Methods in Enzymology: DNA Structures Part A: Synthesis and Physical Analysis of DNA, Academic Press. Each of these general texts is herein incorporated by reference.

In a first aspect, the present invention provides a method of using a vector comprising a transgene to treat a disease or condition of the eye, the method comprising the steps:
(a) administering a solution to a mammalian subject by subretinal injection in an amount effective to at least partially detach the retina to form a subretinal bleb, wherein the solution does not comprise the vector; and
(b) administering a medicament composition by subretinal injection into the bleb formed by step (a), wherein the medicament comprises the vector and is injected in an amount effective to treat the disease or condition;

wherein the transgene is expressible in cells of the mammalian subject.

Structure of the Eye

The method of the present invention is intended for the delivery of a medicament to a mammalian, preferably human eye.

The person skilled in eye surgery will have a detailed and thorough understanding of the structure of the eye. However, the following structures of particular relevance to the present invention are described.

Retina

The retina is the multi-layered membrane which lines the inner posterior chamber of the eye and senses an image of the visual world which is communicated to the brain via the optic nerve. In order from the inside to the outside of the eye, the retina comprises the layers of the neurosensory retina and retinal pigment epithelium, with the choroid lying outside the retinal pigment epithelium.

Neurosensory Retina

The neurosensory retina harbours the photoreceptor cells that directly sense light. It comprises the following layers: internal limiting membrane (ILM); nerve fibre layer; ganglion cell layer; inner plexiform layer; inner nuclear layer; outer plexiform layer; outer nuclear layer (nuclei of the photoreceptors); external limiting membrane (ELM); and photoreceptors (inner and outer segments) of the rods and cones.

Within the neurosensory retina, the fovea is a pit that is composed of closely packed cone cells. This structure is responsible for central high resolution vision.

Retinal Pigment Epithelium

The retinal pigment epithelium (RPE) is a pigmented layer of cells located immediately to the outside of the neurosensory retina. The RPE performs a number of functions, including transport of nutrients and other substances to the photoreceptor cells, and absorption of scattered light to improve vision.

Choroid

The choroid is the vascular layer situated between the RPE and the outer sclera of the eye. The vasculature of the choroid enables provision of oxygen and nutrients to the retina.

Method of Delivering a Medicament

Subretinal Injection

The method of the present invention utilises a number of individual subretinal injection steps. The skilled person will be familiar with and well able to carry out individual subretinal injections.

Subretinal injections are injections into the subretinal space, i.e. underneath the neurosensory retina. During a subretinal injection, the injected material is directed into, and creates a space between, the photoreceptor cell and retinal pigment epithelial (RPE) layers.

When the injection is carried out through a small retinotomy, a retinal detachment may be created. The detached, raised layer of the retina that is generated by the injected material is referred to as a “bleb”.

The hole created by the subretinal injection must be sufficiently small that the injected solution does not significantly reflux back into the vitreous cavity after administration. Such reflux would be particularly problematic when a medicament is injected, because the effects of the medicament would be directed away from the target zone. Preferably, the injection creates a self-sealing entry point in the neurosensory retina, i.e. once the injection needle is removed, the hole created by the needle reseals such that very little or substantially no injected material is released through the hole.

To facilitate this process, specialist subretinal injection needles are commercially available (e.g. DORC 41 G Teflon subretinal injection needle, Dutch Ophthalmic Research Center International BV, Zuidland, The Netherlands). These are needles designed to carry out subretinal injections.

Unless damage to the retina occurs during the injection, and as long as a sufficiently small needle is used, substantially all injected material remains localised between the detached neurosensory retina and the RPE at the site of the localised retinal detachment (i.e. does not reflux into the vitreous cavity). Indeed, the typical persistence of the bleb over a short time frame indicates that there is usually little escape of the injected material into the vitreous. The bleb may dissipate over a longer time frame as the injected material is absorbed.

Visualisations of the eye, in particular the retina, for example using optical coherence tomography, may be made pre-operatively.

Visualising the Retina During Surgery

Under certain circumstances, for example during end-stage retinal degenerations, identifying the retina is difficult because it is thin, transparent and difficult to see against the disrupted and heavily pigmented epithelium on which it sits. The use of a blue vital dye (e.g. Brilliant Peel®, Geuder; MembraneBlue-Dual®, Dorc) may facilitate the identification of the retinal hole made for the retinal detachment procedure (i.e. step (a) in the method of the present invention) so that the medicament can be administered through the same hole without the risk of reflux back into the vitreous cavity.

The use of the blue vital dye also identifies any regions of the retina where there is a thickened internal limiting membrane or epiretinal membrane, as injection through either of these structures would hinder clean access into the subretinal space. Furthermore, contraction of either of these structures in the immediate post-operative period could lead to stretching of the retinal entry hole, which could lead to reflux of the medicament into the vitreous cavity.

The following procedure exemplifies the use of a blue vital dye during subretinal injections. After patients undergo a vitrectomy (with detachment of the posterior hyaloid face in cases where it is still attached), the retina is inspected for any obvious abnormalities. Before performing the two-step procedure of the present invention, a small volume (e.g. 0.05-0.1 mL) of a vital blue dye (e.g. Brilliant Peel® or similar blue membrane stain) is injected directly over the retina in the region to be injected during the method of delivering the medicament. After a brief period to allow staining (e.g. less than 1 min), the blue dye is washed away from the retina and the staining is inspected to identify any breaks in the internal limiting membrane or the presence of an epiretinal membrane. The injection site is selected based on optical coherence tomography (OCT) thickness assessed preoperatively and this area can now be inspected in the presence of the blue dye which will identify any obvious abnormality of the inner retina in this area. An epiretinal membrane could be peeled or the internal limiting membrane peeled back to expose the white retina underneath. This would clear the injection target zone of additional tissue layers that might create a “false” space into which the suspension might be injected.

The blue vital dye staining also helps identify the retinal hole used for the retinal detachment procedure (i.e. step (a) in the method of the present invention) and may be applied again to the same area after membrane removal in order to stain the hole more clearly.

Apparatus

The present invention provides a modified syringe and syringe-loading method for use in the method of delivery of the invention.

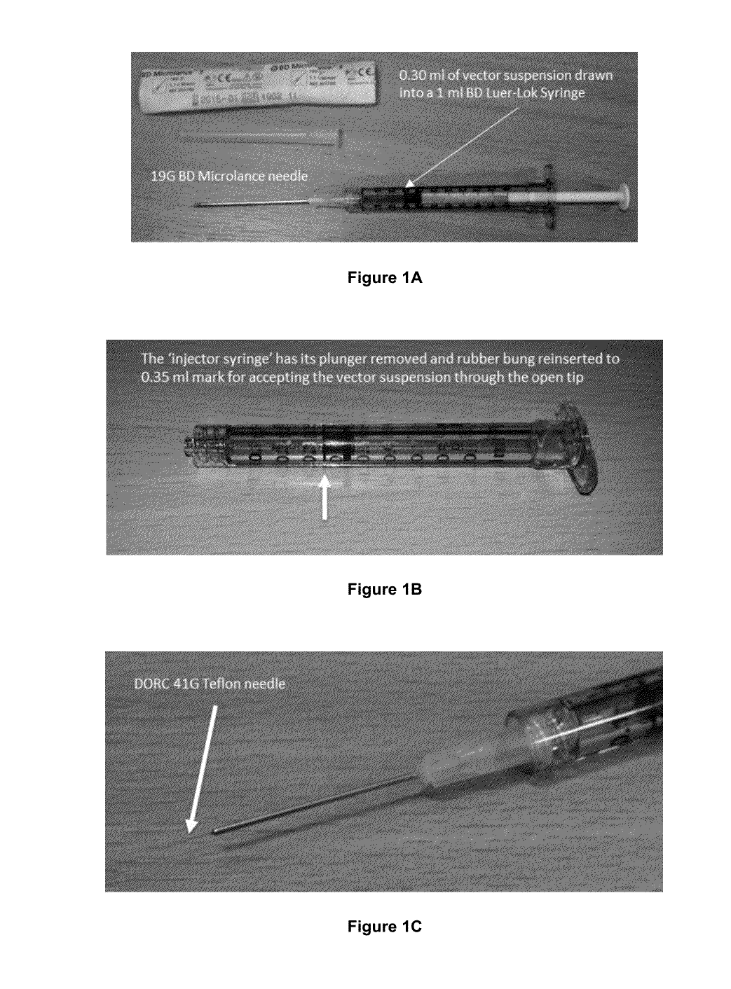

A modified syringe (e.g. 1 mL BD Luer-Lok syringe) may be used for the subretinal injection. This “injector syringe” has had its white plunger removed but the rubber bung reinserted to a pre-determined volume (e.g. the 0.05, 0.10, 0.15, 0.20, 0.25, 0.30, or preferably the 0.35 mL mark) in preparation for loading of the medicament (FIG. 1B). This set-up is necessary for the “injector syringe” to attach to the viscous fluid injection system of the vitrectomy machine.

The medicament suspension will be transferred from a first “drawing-up syringe” to the “injector syringe” by inserting the needle of the “drawing up syringe” through the open tip of the “injector syringe”. Care must be taken to inject slowly so as to avoid bubbles or contact between the needle tip and rubber bung during the loading process.

Once loaded with vector, the “injector syringe” is fitted with a DORC 41 G Teflon subretinal injection needle (Dutch Ophthalmic Research Center International BV, Zuidland, The Netherlands) (FIG. 1C). The 41 G Teflon needle allows self-sealing entry through the neurosensory retina into the subretinal space. Care must be taken not to damage the fine tip. The injector syringe should then be held vertically and tapped gently to clear any bubbles from the suspension.

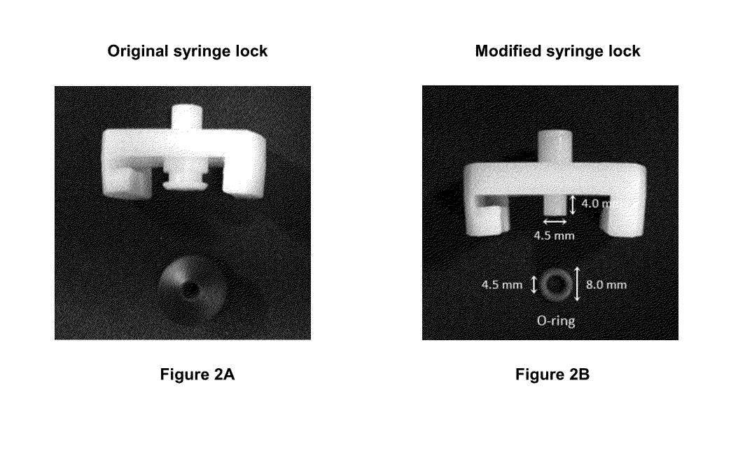

In order to connect the medicament-loaded “injector syringe” to the vitrectomy machine (e.g. Alcon Accurus or Constellation), modification of the standard viscous fluid injection system may be required. A commercially-available syringe lock (e.g. from the Bausch & Lomb Viscous Fluid Pack (CX5710, Bausch & Lomb Inc, NY, USA)) may be custom-modified to fit an 8.0 mm rubber O-ring (as described by FIG. 2). The modified syringe lock should be sterilised before use.

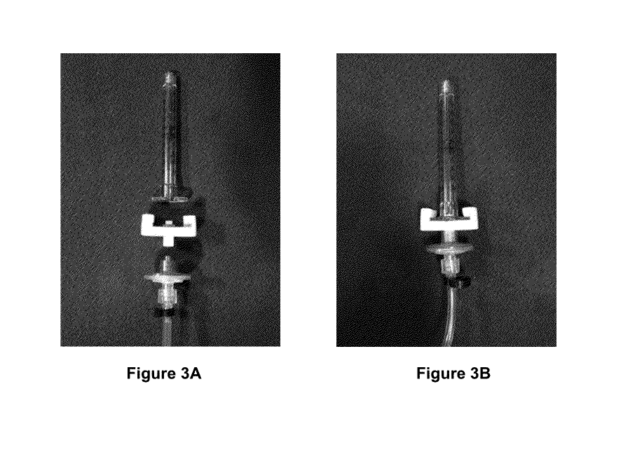

The “injector syringe” may then plug onto the modified syringe lock with an O-ring providing an air-tight seal. The syringe lock, in turn, will connect via a standard Luer-Lok air-filter and tubing (e.g. Easy Connection Tubing (ECT003-00, ALCHIMIA Srl, Ponte S. Nicolò, Italy)) to a vitrectomy machine (e.g. an Alcon Accurus or Constellation vitrectomy machine) (FIG. 3). Versions of the Easy Connection Tubing are commercially available from ALCHIMIA for connecting to other vitrectomy machines.

Injected Solutions and Pharmaceutical Compositions

The medicaments, for example viral vectors, of the present invention may be formulated into pharmaceutical compositions. These compositions may comprise, in addition to the medicament, a pharmaceutically acceptable carrier, diluent, excipient, buffer, stabiliser or other materials well known in the art. Such materials should be non-toxic and should not interfere with the efficacy of the active ingredient. The precise nature of the carrier or other material may be determined by the skilled person according to the route of administration, i.e. subretinal injection.

The pharmaceutical composition is typically in liquid form. Liquid pharmaceutical compositions generally include a liquid carrier such as water, petroleum, animal or vegetable oils, mineral oil or synthetic oil. Physiological saline solution, magnesium chloride, dextrose or other saccharide solution, or glycols such as ethylene glycol, propylene glycol or polyethylene glycol may be included. In some cases, a surfactant, such as pluronic acid (PF68) 0.001% may be used.

For injection at the site of affliction, the active ingredient may be in the form of an aqueous solution which is pyrogen-free, and has suitable pH, isotonicity and stability. The skilled person is well able to prepare suitable solutions using, for example, isotonic vehicles such as Sodium Chloride Injection, Ringer's Injection or Lactated Ringer's Injection. Preservatives, stabilisers, buffers, antioxidants and/or other additives may be included as required.

For delayed release, the medicament may be included in a pharmaceutical composition which is formulated for slow release, such as in microcapsules formed from biocompatible polymers or in liposomal carrier systems according to methods known in the art.

In the method of the present invention, the solution that does not comprise the medicament (i.e. the “first solution” of step (a)) may be similarly formulated to the solution that does comprise the medicament, as described above. A preferred solution that does not comprise the medicament is balanced saline solution (BSS) or a similar buffer solution matched to the pH and osmolality of the subretinal space.

Medicament

The medicament of the present invention is an agent that results in the treatment or prevention of a disease. Preferably the medicament acts on diseases of the eye, for example retinal dystrophies.

The medicament of the present invention may comprise a vector, such as a viral vector. Preferably, the viral vector is in the form of a viral vector particle. The vector may be used for the delivery of a transgene to target cells during gene therapy.

The target cells may, for example, be cells of the neurosensory retina (e.g. photoreceptor cells), retinal pigment epithelium and/or choroid.

Vectors

A vector is a tool that allows or facilitates the transfer of an entity from one environment to another. In accordance with the present invention, and by way of example, some vectors used in recombinant nucleic acid techniques allow entities, such as a segment of nucleic acid (e.g. a heterologous DNA segment, such as a heterologous cDNA segment), to be transferred into a target cell. The vector may serve the purpose of maintaining the heterologous nucleic acid (e.g. DNA or RNA) within the cell, facilitating the replication of the vector comprising a segment of nucleic acid or facilitating the expression of the protein encoded by a segment of nucleic acid.

Vectors may be non-viral or viral. Examples of vectors used in recombinant nucleic acid techniques include, but are not limited to, plasmids, chromosomes, artificial chromosomes and viruses. The vector may also be, for example, a naked nucleic acid (e.g. DNA or RNA). In its simplest form, the vector may itself be a nucleotide of interest.

The vectors used in the invention may be, for example, plasmid or virus vectors and may include a promoter for the expression of a polynucleotide and optionally a regulator of the promoter.

Viral Vectors

In one embodiment, the vector of the present invention is a viral vector. Preferably, the viral vector is in the form of a viral vector particle.

The viral vector may be, for example, an AAV, retroviral, lentiviral or adenoviral vector.

The skilled person is readily able to select a suitable virus for a required purpose as a vector in the present invention, for example based on the size and type of the transgene to be delivered and the type of target cell. Furthermore, methods of preparing and modifying viral vectors and viral vector particles, such as those derived from AAV, retroviruses, lentiviruses or adenoviruses, are well known in the art and can be readily adapted by the skilled person to the required purpose.

AAV Vectors

In a preferred embodiment of the present invention, the viral vector is an AAV vector.

The AAV vector may comprise an AAV genome or a derivative thereof.

An AAV genome is a polynucleotide sequence which encodes functions needed for production of an AAV particle. These functions include those operating in the replication and packaging cycle of AAV in a host cell, including encapsidation of the AAV genome into an AAV viral particle. Naturally occurring AAVs are replication-deficient and rely on the provision of helper functions in trans for completion of a replication and packaging cycle. Accordingly, the AAV genome of the vector of the invention is typically replication-deficient.

The AAV genome may be in single-stranded form, either positive or negative-sense, or alternatively in double-stranded form. The use of a double-stranded form allows bypass of the DNA replication step in the target cell and so can accelerate transgene expression.

The AAV genome may be from any naturally derived serotype, isolate or Glade of AAV. Thus, the AAV genome may be the full genome of a naturally occurring AAV. As is known to the skilled person, AAVs occurring in nature may be classified according to various biological systems.

Commonly, AAVs are referred to in terms of their serotype. A serotype corresponds to a variant subspecies of AAV which, owing to its profile of expression of capsid surface antigens, has a distinctive reactivity which can be used to distinguish it from other variant subspecies. Typically, a virus having a particular AAV serotype does not efficiently cross-react with neutralising antibodies specific for any other AAV serotype.

AAV serotypes include AAV1, AAV2, AAV3, AAV4, AAV5, AAV6, AAV7, AAV8, AAV9, AAV10 and AAV11, and also recombinant serotypes, such as Rec2 and Rec3, recently identified from primate brain. Any of these AAV serotypes may be used in the present invention. Thus, in one embodiment of the present invention, the AAV vector particle is an AAV1, AAV2, AAV3, AAV4, AAV5, AAV6, AAV7, AAV8, AAV9, AAV10, AAV11, Rec2 or Rec3 AAV vector particle.

A preferred serotype of AAV for use in the invention is AAV2.

Other serotypes of particular interest for use in the invention include AAV4, AAV5 and AAV8 which efficiently transduce tissue in the eye, such as the retinal pigment epithelium.

Reviews of AAV serotypes may be found in Choi et al. (2005) Curr. Gene Ther. 5(3): 299-310 and Wu et al. (2006) Molecular Therapy 14(3): 316-27. The sequences of AAV genomes or of elements of AAV genomes including ITR sequences, rep or cap genes for use in the invention may be derived from the following accession numbers for AAV whole genome sequences: Adeno-associated virus 1 NC_002077, AF063497; Adeno-associated virus 2 NC_001401; Adeno-associated virus 3 NC_001729; Adeno-associated virus 3B NC_001863; Adeno-associated virus 4 NC_001829; Adeno-associated virus 5 Y18065, AF085716; Adeno-associated virus 6 NC_001862; Avian AAV ATCC VR-865 AY186198, AY629583, NC_004828; Avian AAV strain DA-1 NC_006263, AY629583; Bovine AAV NC_005889, AY388617.

AAV may also be referred to in terms of clades or clones. This refers to the phylogenetic relationship of naturally derived AAVs, and typically to a phylogenetic group of AAVs which can be traced back to a common ancestor, and includes all descendants thereof. Additionally, AAVs may be referred to in terms of a specific isolate, i.e. a genetic isolate of a specific AAV found in nature. The term genetic isolate describes a population of AAVs which has undergone limited genetic mixing with other naturally occurring AAVs, thereby defining a recognisably distinct population at a genetic level.

The skilled person can select an appropriate serotype, Glade, clone or isolate of AAV for use in the present invention on the basis of their common general knowledge. For instance, the AAV5 capsid has been shown to transduce primate cone photoreceptors efficiently as evidenced by the successful correction of an inherited colour vision defect (Mancuso et al. (2009) Nature 461: 784-7).

It should be understood however that the invention also encompasses use of an AAV genome of other serotypes that may not yet have been identified or characterised.

The AAV serotype determines the tissue specificity of infection (or tropism) of an AAV virus. Accordingly, preferred AAV serotypes for use in AAVs administered to patients in accordance with the invention are those which have natural tropism for or a high efficiency of infection of target cells within the eye. In particular, preferred AAV serotypes for use in the present invention are those which infect cells of the neurosensory retina, retinal pigment epithelium and/or choroid.

Typically, the AAV genome of a naturally derived serotype, isolate or Glade of AAV comprises at least one inverted terminal repeat sequence (ITR). An ITR sequence acts in cis to provide a functional origin of replication and allows for integration and excision of the vector from the genome of a cell. In preferred embodiments, one or more ITR sequences flank the polynucleotide sequence encoding the transgene. The AAV genome typically also comprises packaging genes, such as rep and/or cap genes which encode packaging functions for an AAV particle. The rep gene encodes one or more of the proteins Rep78, Rep68, Rep52 and Rep40 or variants thereof. The cap gene encodes one or more capsid proteins such as VP1, VP2 and VP3 or variants thereof. These proteins make up the capsid of an AAV particle. Capsid variants are discussed below.

A promoter will be operably linked to each of the packaging genes. Specific examples of such promoters include the p5, p19 and p40 promoters (Laughlin et al. (1979) Proc. Natl. Acad. Sci. USA 76: 5567-71). For example, the p5 and p19 promoters are generally used to express the rep gene, while the p40 promoter is generally used to express the cap gene.

As discussed above, the AAV genome used in the vector of the invention may therefore be the full genome of a naturally occurring AAV. For example, a vector comprising a full AAV genome may be used to prepare an AAV vector or vector particle in vitro. However, while such a vector may in principle be administered to patients, this will rarely be done in practice. Preferably the AAV genome will be derivatised for the purpose of administration to patients. Such derivatisation is standard in the art and the present invention encompasses the use of any known derivative of an AAV genome, and derivatives which could be generated by applying techniques known in the art. Derivatisation of the AAV genome and of the AAV capsid are reviewed in Coura and Nardi (2007) Virology Journal 4: 99, and in Choi et al. and Wu et al., referenced above.

Derivatives of an AAV genome include any truncated or modified forms of an AAV genome which allow for expression of a transgene from a vector of the invention in vivo. Typically, it is possible to truncate the AAV genome significantly to include minimal viral sequence yet retain the above function. This is preferred for safety reasons to reduce the risk of recombination of the vector with wild-type virus, and also to avoid triggering a cellular immune response by the presence of viral gene proteins in the target cell.

Typically, a derivative will include at least one inverted terminal repeat sequence (ITR), preferably more than one ITR, such as two ITRs or more. One or more of the ITRs may be derived from AAV genomes having different serotypes, or may be a chimeric or mutant ITR. A preferred mutant ITR is one having a deletion of a trs (terminal resolution site). This deletion allows for continued replication of the genome to generate a single-stranded genome which contains both coding and complementary sequences, i.e. a self-complementary AAV genome. This allows for bypass of DNA replication in the target cell, and so enables accelerated transgene expression.

The one or more ITRs will preferably flank the polynucleotide sequence encoding the transgene at either end. The inclusion of one or more ITRs is preferred to aid concatamer formation of the vector of the invention in the nucleus of a host cell, for example following the conversion of single-stranded vector DNA into double-stranded DNA by the action of host cell DNA polymerases. The formation of such episomal concatamers protects the vector construct during the life of the host cell, thereby allowing for prolonged expression of the transgene in vivo.

In preferred embodiments, ITR elements will be the only sequences retained from the native AAV genome in the derivative. Thus, a derivative will preferably not include the rep and/or cap genes of the native genome and any other sequences of the native genome. This is preferred for the reasons described above, and also to reduce the possibility of integration of the vector into the host cell genome. Additionally, reducing the size of the AAV genome allows for increased flexibility in incorporating other sequence elements (such as regulatory elements) within the vector in addition to the transgene.

The following portions could therefore be removed in a derivative of the invention: one inverted terminal repeat (ITR) sequence, the replication (rep) and capsid (cap) genes (NB: the rep gene in the wild type AAV genome should not to be confused with REP1, the human gene affected in choroideremia, and an example transgene for use in the present invention). However, in some embodiments, derivatives may additionally include one or more rep and/or cap genes or other viral sequences of an AAV genome. Naturally occurring AAV integrates with a high frequency at a specific site on human chromosome 19, and shows a negligible frequency of random integration, such that retention of an integrative capacity in the vector may be tolerated in a therapeutic setting.

Where a derivative comprises capsid proteins i.e. VP1, VP2 and/or VP3, the derivative may be a chimeric, shuffled or capsid-modified derivative of one or more naturally occurring AAVs. In particular, the invention encompasses the provision of capsid protein sequences from different serotypes, clades, clones, or isolates of AAV within the same vector i.e. pseudotyping.

Chimeric, shuffled or capsid-modified derivatives will be typically selected to provide one or more desired functionalities for the viral vector. Thus, these derivatives may display increased efficiency of gene delivery, decreased immunogenicity (humoral or cellular), an altered tropism range and/or improved targeting of a particular cell type compared to an AAV vector comprising a naturally occurring AAV genome, such as that of AAV2. Increased efficiency of gene delivery may be effected by improved receptor or co-receptor binding at the cell surface, improved internalisation, improved trafficking within the cell and into the nucleus, improved uncoating of the viral particle and improved conversion of a single-stranded genome to double-stranded form. Increased efficiency may also relate to an altered tropism range or targeting of a specific cell population, such that the vector dose is not diluted by administration to tissues where it is not needed.

Chimeric capsid proteins include those generated by recombination between two or more capsid coding sequences of naturally occurring AAV serotypes. This may be performed for example by a marker rescue approach in which non-infectious capsid sequences of one serotype are co-transfected with capsid sequences of a different serotype, and directed selection is used to select for capsid sequences having desired properties. The capsid sequences of the different serotypes can be altered by homologous recombination within the cell to produce novel chimeric capsid proteins.

Chimeric capsid proteins also include those generated by engineering of capsid protein sequences to transfer specific capsid protein domains, surface loops or specific amino acid residues between two or more capsid proteins, for example between two or more capsid proteins of different serotypes.

Shuffled or chimeric capsid proteins may also be generated by DNA shuffling or by error-prone PCR. Hybrid AAV capsid genes can be created by randomly fragmenting the sequences of related AAV genes e.g. those encoding capsid proteins of multiple different serotypes and then subsequently reassembling the fragments in a self-priming polymerase reaction, which may also cause crossovers in regions of sequence homology. A library of hybrid AAV genes created in this way by shuffling the capsid genes of several serotypes can be screened to identify viral clones having a desired functionality. Similarly, error prone PCR may be used to randomly mutate AAV capsid genes to create a diverse library of variants which may then be selected for a desired property.

The sequences of the capsid genes may also be genetically modified to introduce specific deletions, substitutions or insertions with respect to the native wild-type sequence. In particular, capsid genes may be modified by the insertion of a sequence of an unrelated protein or peptide within an open reading frame of a capsid coding sequence, or at the N- and/or C-terminus of a capsid coding sequence.

The unrelated protein or peptide may advantageously be one which acts as a ligand for a particular cell type, thereby conferring improved binding to a target cell or improving the specificity of targeting of the vector to a particular cell population. An example might include the use of RGD peptide to block uptake in the retinal pigment epithelium and thereby enhance transduction of surrounding retinal tissues (Cronin et al. (2008) ARVO Abstract: D1048). The unrelated protein may also be one which assists purification of the viral particle as part of the production process i.e. an epitope or affinity tag. The site of insertion will typically be selected so as not to interfere with other functions of the viral particle e.g. internalisation, trafficking of the viral particle. The skilled person can identify suitable sites for insertion based on their common general knowledge. Particular sites are disclosed in Choi et al., referenced above.

The invention additionally encompasses the provision of sequences of an AAV genome in a different order and configuration to that of a native AAV genome. The invention also encompasses the replacement of one or more AAV sequences or genes with sequences from another virus or with chimeric genes composed of sequences from more than one virus. Such chimeric genes may be composed of sequences from two or more related viral proteins of different viral species.

The vector of the invention may take the form of a polynucleotide sequence comprising an AAV genome or derivative thereof and a sequence encoding a transgene or a variant thereof.

The AAV particles of the invention include transcapsidated forms wherein an AAV genome or derivative having an ITR of one serotype is packaged in the capsid of a different serotype. The AAV particles of the invention also include mosaic forms wherein a mixture of unmodified capsid proteins from two or more different serotypes makes up the viral capsid. The AAV particle also includes chemically modified forms bearing ligands adsorbed to the capsid surface. For example, such ligands may include antibodies for targeting a particular cell surface receptor.

Thus, for example, the AAV particles of the invention include those with an AAV2 genome and AAV2 capsid proteins (AAV2/2), those with an AAV2 genome and AAV5 capsid proteins (AAV2/5) and those with an AAV2 genome and AAV8 capsid proteins (AAV2/8).

Retro Viral and Lentiviral Vectors

In another embodiment of the present invention, the viral vector is a retroviral vector.

Retroviruses and lentiviruses have been adapted for use as gene therapy vectors for a wide range of purposes.

Retroviruses may be broadly divided into two categories, “simple” and “complex”. Retroviruses may even be further divided into seven groups. Five of these groups represent retroviruses with oncogenic potential. The remaining two groups are the lentiviruses and the spumaviruses. A review of these retroviruses is presented in Coffin, J. M. et al. (1997) Retroviruses, pp. 758-763, Cold Spring Harbor Laboratory Press, Eds: J. M. Coffin, S. M. Hughes, H. E. Varmus.

The retroviral vector used in the present invention may be derived from or may be derivable from any suitable retrovirus. A large number of different retroviruses have been identified and the skilled person is well able to select a suitable retrovirus for a particular purpose. Examples include: murine leukemia virus (MLV), human T-cell leukaemia virus (HTLV), mouse mammary tumour virus (MMTV), Rous sarcoma virus (RSV), Fujinami sarcoma virus (FuSV), Moloney murine leukemia virus (MoMLV), FBR murine osteosarcoma virus (FBR MSV), Moloney murine sarcoma virus (Mo-MSV), Abelson murine leukemia virus (A-MLV), Avian myelocytomatosis virus-29 (MC29) and Avian erythroblastosis virus (AEV). A detailed list of retroviruses may be found in Coffin, J. M. et al. (1997) Retroviruses, pp. 758-763, Cold Spring Harbor Laboratory Press, Eds: J. M. Coffin, S. M. Hughes, H. E. Varmus.

In another embodiment of the present invention, the viral vector is a lentiviral vector.

Lentivirus vectors are part of the larger group of retroviral vectors. A detailed list of lentiviruses may be found in Coffin, J. M. et al. (1997) Retroviruses, pp. 758-763, Cold Spring Harbor Laboratory Press, Eds: J. M. Coffin, S. M. Hughes, H. E. Varmus. In brief, lentiviruses can be divided into primate and non-primate groups. Examples of primate lentiviruses include but are not limited to: the human immunodeficiency virus (HIV), the causative agent of human auto-immunodeficiency syndrome (AIDS); and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV), and the more recently described feline immunodeficiency virus (FIV) and bovine immunodeficiency virus (BIV).

Adenoviral Vectors

In another embodiment of the present invention, the viral vector is an adenovirus vector.

The adenovirus is a double-stranded, linear DNA virus that does not go through an RNA intermediate. There are over 50 different human serotypes of adenovirus divided into 6 subgroups based on the genetic sequence homology. The natural targets of adenovirus are the respiratory and gastrointestinal epithelia, generally giving rise to only mild symptoms. Serotypes 2 and 5 (with 95% sequence homology) are most commonly used in adenoviral vector systems and are normally associated with upper respiratory tract infections in the young.

Adenoviruses have been used as vectors for gene therapy and for expression of heterologous genes. The large (36 kb) genome can accommodate up to 8 kb of foreign insert DNA and is able to replicate efficiently in complementing cell lines to produce very high titres of up to 10¹². Adenovirus is thus one of the best systems to study the expression of genes in primary non-replicative cells.

The expression of viral or foreign genes from the adenovirus genome does not require a replicating cell. Adenoviral vectors enter cells by receptor mediated endocytosis. Once inside the cell, adenovirus vectors rarely integrate into the host chromosome. Instead, they function episomally (independently from the host genome) as a linear genome in the host nucleus. Hence the use of recombinant adenovirus alleviates the problems associated with random integration into the host genome.

Method of Treatment

It is to be appreciated that all references herein to treatment include curative, palliative and prophylactic treatment; although in the context of the present invention references to preventing are more commonly associated with prophylactic treatment. Treatment may also include arresting progression in the severity of a disease.

The treatment of mammals, particularly humans, is preferred. However, both human and veterinary treatments are within the scope of the present invention.

Transgenes

The method of the present invention may be used to deliver transgenes to target cells during gene therapy. For example, the method may be used to deliver viral vectors comprising a transgene to target cells in the eye.

The transgene may give rise to a therapeutic effect. The transgene may, for example, supplement a defective or absent gene with a functional copy of that gene, inactivate an improperly functioning gene or introduce a new therapeutic gene to the target cell.

Suitable transgenes include, but are not limited to sequences encoding enzymes, cytokines, chemokines, hormones, antibodies, anti-oxidant molecules, engineered immunoglobulin-like molecules, single chain antibodies, fusion proteins, immune co-stimulatory molecules, immunomodulatory molecules, anti-sense RNA, microRNA, shRNA, siRNA, ribozymes, miRNA target sequences, a transdomain negative mutants of a target proteins, toxins, conditional toxins, antigens, tumour suppressor proteins, growth factors, transcription factors, membrane proteins, surface receptors, anti-cancer molecules, vasoactive proteins and peptides, anti-viral proteins and ribozymes, and derivatives thereof (such as derivatives with an associated reporter group). The transgenes may also encode pro-drug activating enzymes.

Suitable transgenes for use in the present invention include genes encoding REP1 (i.e. the gene encoding Rab escort protein 1, REP1); retinal pigment epithelium-specific 65 kDa protein (RPE65); arylhydrocarbon-interacting receptor protein like 1 (AIPL1); crumbs homologue 1 (CRB1); lecithin retinal acetyltransferase (LRAT); photoreceptor-specific homeo box (CRX); retinal guanylate cyclase gene (GUCY2D); RPGR Interacting Protein 1 (RPGRIP1); dystrophin; ATP-binding cassette, sub-family A (ABC1), member 4 (ABCR); epithelial membrane protein 1 (EMP1); TIMP metallopeptidase inhibitor 3 (TIMP3); MERTCK and ELOVL fatty acid elongase 4 (ELOVL4).

REP1

In one embodiment of the present invention, the transgene is the Rab escort protein 1 (REP1) gene.

Rab escort protein 1 may also be known as Rab protein geranylgeranyltransferase component A. Furthermore, the REP1 gene may sometimes be known as the CHM gene.

In one embodiment, the amino acid sequence of the human REP1 protein is:

(SEQ ID NO: 1)
MADTLPSEFDVIVIGTGLPESIIAAACSRSGRRVLHVDSRSYYGGNWASF
SFSGLLSWLKEYQENSDIVSDSPVWQDQILENEEAIALSRKDKTIQHVEV
FCYASQDLHEDVEEAGALQKNHALVTSANSTEAADSAFLPTEDESLSTMS
CEMLTEQTPSSDPENALEVNGAEVTGEKENHCDDKTCVPSTSAEDMSENV
PIAEDTTEQPKKNRITYSQIIKEGRRFNIDLVSKLLYSRGLLIDLLIKSN
VSRYAEFKNITRILAFREGRVEQVPCSRADVFNSKQLTMVEKRMLMKFLT
FCMEYEKYPDEYKGYEEITFYEYLKTQKLTPNLQYIVMHSIAMTSETASS
TIDGLKATKNFLHCLGRYGNTPFLFPLYGQGELPQCFCRMCAVFGGIYCL
RHSVQCLVVDKESRKCKAIIDQFGQRIISEHFLVEDSYFPENMCSRVQYR
QISRAVLITDRSVLKTDSDQQISILTVPAEEPGTFAVRVIELCSSTMTCM
KGTYLVHLTCTSSKTAREDLESVVQKLFVPYTEMEIENEQVEKPRILWAL
YFNMRDSSDISRSCYNDLPSNVYVCSGPDCGLGNDNAVKQAETLFQEICP
NEDFCPPPPNPEDIILDGDSLQPEASESSAIPEANSETFKESTNLGNLEE
SSE

In one embodiment, the human cDNA sequence for REP1 is:

(SEQ ID NO: 2)
atggcggatactctcccttcggagtttgatgtgatcgtaatagggacggg
tttgcctgaatccatcattgcagctgcatgttcaagaagtggccggagag
ttctgcatgttgattcaagaagctactatggaggaaactgggccagtttt
agcttttcaggactattgtcctggctaaaggaataccaggaaaacagtga
cattgtaagtgacagtccagtgtggcaagaccagatccttgaaaatgaag
aagccattgctcttagcaggaaggacaaaactattcaacatgtggaagta
ttttgttatgccagtcaggatttgcatgaagatgtcgaagaagctggtgc
actgcagaaaaatcatgctcttgtgacatctgcaaactccacagaagctg
cagattctgccttcctgcctacggaggatgagtcattaagcactatgagc
tgtgaaatgctcacagaacaaactccaagcagcgatccagagaatgcgct
agaagtaaatggtgctgaagtgacaggggaaaaagaaaaccattgtgatg
ataaaacttgtgtgccatcaacttcagcagaagacatgagtgaaaatgtg
cctatagcagaagataccacagagcaaccaaagaaaaacagaattactta
ctcacaaattattaaagaaggcaggagatttaatattgatttagtatcaa
agctgctgtattctcgaggattactaattgatcttctaatcaaatctaat
gttagtcgatatgcagagtttaaaaatattaccaggattcttgcatttcg
agaaggacgagtggaacaggttccgtgttccagagcagatgtctttaata
gcaaacaacttactatggtagaaaagcgaatgctaatgaaatttcttaca
ttttgtatggaatatgagaaatatcctgatgaatataaaggatatgaaga
gatcacattttatgaatatttaaagactcaaaaattaacccccaacctcc
aatatattgtcatgcattcaattgcaatgacatcagagacagccagcagc
accatagatggtctcaaagctaccaaaaactttcttcactgtcttgggcg
gtatggcaacactccatttttgtttcctttatatggccaaggagaactcc
cccagtgtttctgcaggatgtgtgctgtgtttggtggaatttattgtctt
cgccattcagtacagtgccttgtagtggacaaagaatccagaaaatgtaa
agcaattatagatcagtttggtcagagaataatctctgagcatttcctcg
tggaggacagttactttcctgagaacatgtgctcacgtgtgcaatacagg
cagatctccagggcagtgctgattacagatagatctgtcctaaaaacaga
ttcagatcaacagatttccattttgacagtgccagcagaggaaccaggaa
cttttgctgttcgggtcattgagttatgttcttcaacgatgacatgcatg
aaaggcacctatttggttcatttgacttgcacatcttctaaaacagcaag
agaagatttagaatcagttgtgcagaaattgtttgttccatatactgaaa
tggagatagaaaatgaacaagtagaaaagccaagaattctgtgggctctt
tacttcaatatgagagattcgtcagacatcagcaggagctgttataatga
tttaccatccaacgtttatgtctgctctggcccagattgtggtttaggaa
atgataatgcagtcaaacaggctgaaacacttttccaggaaatctgcccc
aatgaagatttctgtccccctccaccaaatcctgaagacattatccttga
tggagacagtttacagccagaggcttcagaatccagtgccataccagagg
ctaactcggagactttcaaggaaagcacaaaccttggaaacctagaggag
tcctctgaataa

A further cDNA sequence of REP1 is:

(SEQ ID NO: 3)
gatatcgaattcctgcagcccggcggcaccatggcggatactctcccttc
ggagtttgatgtgatcgtaatagggacgggtttgcctgaatccatcattg
cagctgcatgttcaagaagtggccggagagttctgcatgttgattcaaga
agctactatggaggaaactgggccagttttagcttttcaggactattgtc
ctggctaaaggaataccaggaaaacagtgacattgtaagtgacagtccag
tgtggcaagaccagatccttgaaaatgaagaagccattgctcttagcagg
aaggacaaaactattcaacatgtggaagtattttgttatgccagtcagga
tttgcatgaagatgtcgaagaagctggtgcactgcagaaaaatcatgctc
ttgtgacatctgcaaactccacagaagctgcagattctgccttcctgcct
acggaggatgagtcattaagcactatgagctgtgaaatgctcacagaaca
aactccaagcagcgatccagagaatgcgctagaagtaaatggtgctgaag
tgacaggggaaaaagaaaaccattgtgatgataaaacttgtgtgccatca
acttcagcagaagacatgagtgaaaatgtgcctatagcagaagataccac
agagcaaccaaagaaaaacagaattacttactcacaaattattaaagaag
gcaggagatttaatattgatttagtatcaaagctgctgtattctcgagga
ttactaattgatcttctaatcaaatctaatgttagtcgatatgcagagtt
taaaaatattaccaggattcttgcatttcgagaaggacgagtggaacagg
ttccgtgttccagagcagatgtctttaatagcaaacaacttactatggta
gaaaagcgaatgctaatgaaatttcttacattttgtatggaatatgagaa
atatcctgatgaatataaaggatatgaagagatcacattttatgaatatt
taaagactcaaaaattaacccccaacctccaatatattgtcatgcattca
attgcaatgacatcagagacagccagcagcaccatagatggtctcaaagc
taccaaaaactttcttcactgtcttgggcggtatggcaacactccatttt
tgtttcctttatatggccaaggagaactcccccagtgtttctgcaggatg
tgtgctgtgtttggtggaatttattgtcttcgccattcagtacagtgcct
tgtagtggacaaagaatccagaaaatgtaaagcaattatagatcagtttg
gtcagagaataatctctgagcatttcctcgtggaggacagttactttcct
gagaacatgtgctcacgtgtgcaatacaggcagatctccagggcagtgct
gattacagatagatctgtcctaaaaacagattcagatcaacagatttcca
ttttgacagtgccagcagaggaaccaggaacttttgctgttcgggtcatt
gagttatgttcttcaacgatgacatgcatgaaaggcacctatttggttca
tttgacttgcacatcttctaaaacagcaagagaagatttagaatcagttg
tgcagaaattgtttgttccatatactgaaatggagatagaaaatgaacaa
gtagaaaagccaagaattctgtgggctctttacttcaatatgagagattc
gtcagacatcagcaggagctgttataatgatttaccatccaacgtttatg
tctgctctggcccagattgtggtttaggaaatgataatgcagtcaaacag
gctgaaacacttttccaggaaatctgccccaatgaagatttctgtccccc
tccaccaaatcctgaagacattatccttgatggagacagtttacagccag
aggcttcagaatccagtgccataccagaggctaactcggagactttcaag
gaaagcacaaaccttggaaacctagaggagtcctctgaataa

A REP1 protein or variant thereof is any polypeptide which assists in prenylation of a Rab GTPase protein. The ability of a REP1 polypeptide or variant thereof to assist in prenylation of a Rab GTPase protein can be routinely determined by a person skilled in the art. A polynucleotide sequence encoding a variant of REP1 is any sequence which encodes a protein assisting in prenylation activity for a Rab-1 GTPase. Preferably the sequence encodes a protein which assists in providing similar or higher prenylation activity for Rab-1 GTPase compared to the protein of SEQ ID NO: 1.

The transgene of the present invention may, for example, encode an amino acid sequence that has at least 50%, 60%, 70%, 80%, 90%, 95%, 99% or 100% identity to SEQ ID NO: 1, wherein the amino acid sequence substantially retains the natural function of the protein represented by SEQ ID NO: 1.

The transgene of the present invention may, for example, comprise a nucleotide sequence that has at least 50%, 60%, 70%, 80%, 90%, 95%, 99% or 100% identity to SEQ ID NO: 2 or 3, wherein the protein encoded by the nucleotide sequence substantially retains the natural function of the protein represented by SEQ ID NO: 1.

Suitable variants of REP1 are described further in WO 2012/114090.

RPE65

In one embodiment of the present invention, the transgene is the retinal pigment epithelium-specific 65 kDa protein (RPE65) gene.

In one embodiment, the amino acid sequence of the human RPE65 protein is:

(SEQ ID NO: 4)
MSIQVEHPAGGYKKLFETVEELSSPLTAHVTGRIPLWLTGSLLRCGPGLF
EVGSEPFYHLFDGQALLHKFDFKEGHVTYHRRFIRTDAYVRAMTEKRIVI
TEFGTCAFPDPCKNIFSRFFSYFRGVEVTDNALVNVYPVGEDYYACTETN
FITKINPETLETIKQVDLCNYVSVNGATAHPHIENDGTVYNIGNCFGKNF
SIAYNIVKIPPLQADKEDPISKSEIVVQFPCSDRFKPSYVHSFGLTPNYI
VFVETPVKINLFKFLSSWSLWGANYMDCFESNETMGVWLHIADKKRKKYL
NNKYRTSPFNLFHHINTYEDNGFLIVDLCCWKGFEFVYNYLYLANLRENW
EEVKKNARKAPQPEVRRYVLPLNIDKADTGKNLVTLPNTTATAILCSDET
IWLEPEVLFSGPRQAFEFPQINYQKYCGKPYTYAYGLGLNHFVPDRLCKL
NVKTKETWVWQEPDSYPSEPIFVSHPDALEEDDGVVLSVVVSPGAGQKPA
YLLILNAKDLSEVARAEVEINIPVTFHGLFKKS

The transgene of the present invention may, for example, encode an amino acid sequence that has at least 50%, 60%, 70%, 80%, 90%, 95%, 99% or 100% identity to SEQ ID NO: 4, wherein the amino acid sequence substantially retains the natural function of the protein represented by SEQ ID NO: 4.

Promoters and Regulatory Sequences

The vector of the invention may also include elements allowing for the expression of the transgene in vitro or in vivo. These may be referred to as expression control sequences. Thus, the vector typically comprises expression control sequences (e.g. comprising a promoter sequence) operably linked to the polynucleotide sequence encoding the transgene.

By “operably linked”, it is to be understood that the individual components are linked together in a manner which enables them to carry out their function (e.g. promoting expression of the transgene in a cell) substantially unhindered.

Any suitable promoter may be used, the selection of which may be readily made by the skilled person. The promoter sequence may be constitutively active (i.e. operational in any host cell background), or alternatively may be active only in a specific host cell environment, thus allowing for targeted expression of the transgene in a particular cell type (e.g. a tissue-specific promoter). The promoter may show inducible expression in response to presence of another factor, for example a factor present in a host cell. In any event, where the vector is administered for therapy, it is preferred that the promoter should be functional in the target cell background.

In some embodiments, it is preferred that the promoter shows retinal-cell specific expression in order to allow for the transgene to only be expressed in retinal cell populations. Thus, expression from the promoter may be retinal-cell specific, for example confined only to cells of the neurosensory retina and retinal pigment epithelium.

Preferred promoters include the chicken beta-actin (CBA) promoter, optionally in combination with a cytomegalovirus (CME) enhancer element. A particularly preferred promoter is a hybrid CBA/CAG promoter, for example the promoter used in the rAVE expression cassette (GeneDetect.com). A further preferred promoter has the sequence:

(SEQ ID NO: 5)
attgacgtcaataatgacgtatgttcccatagtaacgccaatagggactt
tccattgacgtcaatgggtggagtatttacggtaaactgcccacttggca
gtacatcaagtgtatcatatgccaagtacgccccctattgacgtcaatga
cggtaaatggcccgcctggcattatgcccagtacatgaccttatgggact
ttcctacttggcagtacatctacgtattagtcatcgctattaccatggtc
gaggtgagccccacgttctgcttcactctccccatctcccccccctcccc
acccccaattttgtatttatttattttttaattattttgtgcagcgatgg
gggcggggggggggggggggcgcgcgccaggcggggcggggcggggcgag
gggcggggcggggcgaggcggagaggtgcggcggcagccaatcagagcgg
cgcgctccgaaagtttccttttatggcgaggcggcggcggcggcggccct
ataaaaagcgaagcgcgcggcgggcgggagtcgctgcgcgctgccttcgc
cccgtgccccgctccgccgccgcctcgcgccgcccgccccggctctgact
gaccgcgttactcccacaggtgagcgggcgggacggcccttctcctccgg
gctgtaattagcgcttggtttaatgacggcttgtttcttttctgtggctg
cgtgaaagccttgaggggctccgggagggccctttgtgcggggggagcgg
ctcggggctgtccgcggggggacggctgccttcgggggggacggggcagg
gcggggttcggcttctggcgtgtgaccggcggctctagagcctctgctaa
ccatgttcatgccttcttctttttcctacagctcctgggcaacgtgctgg
ttattgtgctgtctcatcattttggcaaagaatt

Examples of promoters based on human sequences that would induce retina-specific gene expression include rhodospin kinase for rods and cones (Allocca et al. (2007) J. Virol. 81: 11372-80), PR2.1 for cones only (Mancuso et al. (2009) Nature 461: 784-7) and/or RPE65 for the retinal pigment epithelium (Bainbridge et al. (2008) N. Engl. J. Med. 358: 2231-9).

The vector of the invention may also comprise one or more additional regulatory sequences with may act pre- or post-transcriptionally. The regulatory sequence may be part of the native transgene locus or may be a heterologous regulatory sequence. The vector of the invention may comprise portions of the 5′-UTR or 3′-UTR from the native transgene transcript.

Regulatory sequences are any sequences which facilitate expression of the transgene, i.e. act to increase expression of a transcript, improve nuclear export of mRNA or enhance its stability. Such regulatory sequences include for example enhancer elements, postregulatory elements and polyadenylation sites. A preferred polyadenylation site is the Bovine Growth Hormone poly-A signal which may be as shown below:

(SEQ ID NO: 6)
tcgctgatcagcctcgactgtgccttctagttgccagccatctgttgttt
gcccctcccccgtgccttccttgaccctggaaggtgccactcccactgtc
ctttcctaataaaatgaggaaattgcatcgcattgtctgagtaggtgtca
ttctattctggggggtggggtggggcaggacagcaagggggaggattggg
aagacaatagcaggcatgctggggatgcggtgggctctatggcttctgag
gcggaaagaaccagctgggg

In the context of the vector of the invention such regulatory sequences will be cis-acting. However, the invention also encompasses the use of trans-acting regulatory sequences located on additional genetic constructs.

A preferred post-regulatory element for use in a vector of the invention is the woodchuck hepatitis postregulatory element (WPRE) or a variant thereof. The sequence of the WPRE is shown below:

(SEQ ID NO: 7)
atcaacctctggattacaaaatttgtgaaagattgactggtattcttaac
tatgttgctccttttacgctatgtggatacgctgctttaatgcctttgta
tcatgctattgcttcccgtatggctttcattttctcctccttgtataaat
cctggttgctgtctctttatgaggagttgtggcccgttgtcaggcaacgt
ggcgtggtgtgcactgtgtttgctgacgcaacccccactggttggggcat
tgccaccacctgtcagctcctttccgggactttcgctttccccctcccta
ttgccacggcggaactcatcgccgcctgccttgcccgctgctggacaggg
gctcggctgttgggcactgacaattccgtggtgttgtcggggaaatcatc
gtcctttccttggctgctcgcctgtgttgccacctggattctgcgcggga
cgtccttctgctacgtcccttcggccctcaatccagcggaccttccttcc
cgcggcctgctgccggctctgcggcctcttccgcgtcttcgccttcgccc
tcagacgagtcggatctccctttgggccgcctccccgc

The invention encompasses the use of any variant sequence of the WPRE which increases expression of the transgene compared to a vector without a WPRE. Preferably, variant sequences display at least 70% homology to SEQ ID NO: 7 over its entire sequence, more preferably 75%, 80%, 85%, 90% and more preferably at least 95%, 97% or 99% homology to SEQ ID NO: 7 over its entire sequence.

Another regulatory sequence which may be used in a vector of the present invention is a scaffold-attachment region (SAR). Additional regulatory sequences may be readily selected by the skilled person.

Diseases

The method of the present invention is widely applicable to the treatment of diseases of the eye. Preferably the disease affects the back of the eye, for example the retina.

In one embodiment, the method is for treating a retinal dystrophy.

In another embodiment, the method is for treating choroideremia, Leber congenital amaurosis, cone-rod dystrophy, macular dystrophy, cone dystrophy, achromatopsia, retinitis pigmentosa or age-related macular degeneration.

In a preferred embodiment, the method is for treating choroideremia.

Choroideremia

Choroideremia is a rare X-linked progressive degeneration of the choroid, retinal pigment epithelium and photoreceptors of the eye. The typical natural history in afflicted males is onset of nightblindness during teenage years, and then progressive loss of peripheral vision during the 20's and 30's leading to complete blindness in the 40's. Female carriers have mild symptoms most notably nightblindness, but may occasionally have a more severe phenotype.

Choroideremia is caused by mutations in the REP1 gene, (Rab escort protein-1), which is located on the X chromosome 21q region. In most cells in the body, the REP2 protein, which is 75% homologous to REP1, compensates for the REP1 deficiency. In the eye, however, for reasons that are not yet clear, REP2 is unable to compensate for the REP1 deficiency. Hence in the eye, REP polypeptide activity is insufficient to maintain normal prenylation of the target proteins (Rab GTPases) leading to cellular dysfunction and ultimate death, primarily affecting the outer retina and choroid.

Choroideremia may be successfully treated by providing functional copies of the REP1 transgene to the affected cells of the eye (MacLaren, R. E. et al. (2014) Lancet 383: 1129-37).

Variants, Derivatives, Analogues, Homologues and Fragments

In addition to the specific proteins and nucleotides mentioned herein, the present invention also encompasses the use of variants, derivatives, analogues, homologues and fragments thereof.

In the context of the present invention, a variant of any given sequence is a sequence in which the specific sequence of residues (whether amino acid or nucleic acid residues) has been modified in such a manner that the polypeptide or polynucleotide in question substantially retains at least one of its endogenous functions. A variant sequence can be obtained by addition, deletion, substitution, modification, replacement and/or variation of at least one residue present in the naturally-occurring protein.

The term “derivative” as used herein, in relation to proteins or polypeptides of the present invention includes any substitution of, variation of, modification of, replacement of, deletion of and/or addition of one (or more) amino acid residues from or to the sequence providing that the resultant protein or polypeptide substantially retains at least one of its endogenous functions.

The term “analogue” as used herein, in relation to polypeptides or polynucleotides includes any mimetic, that is, a chemical compound that possesses at least one of the endogenous functions of the polypeptides or polynucleotides which it mimics.

Typically, amino acid substitutions may be made, for example from 1, 2 or 3 to 10 or 20 substitutions provided that the modified sequence substantially retains the required activity or ability. Amino acid substitutions may include the use of non-naturally occurring analogues.

Proteins used in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R H |
| AROMATIC | | F W Y |

The term “homologue” as used herein means an entity having a certain homology with the wild type amino acid sequence and the wild type nucleotide sequence. The term “homology” can be equated with “identity”.

A homologous sequence may include an amino acid sequence which may be at least 50%, 55%, 60%, 65%, 70%, 75%, 80%, 85% or 90% identical, preferably at least 95% or 97% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

A homologous sequence may include a nucleotide sequence which may be at least 50%, 55%, 60%, 65%, 70%, 75%, 80%, 85% or 90% identical, preferably at least 95% or 97% or 99% identical to the subject sequence. Although homology can also be considered in terms of similarity, in the context of the present invention it is preferred to express homology in terms of sequence identity.

Preferably, reference to a sequence which has a percent identity to any one of the SEQ ID NOs detailed herein refers to a sequence which has the stated percent identity over the entire length of the SEQ ID NO referred to.

Homology comparisons can be conducted by eye or, more usually, with the aid of readily available sequence comparison programs. These commercially available computer programs can calculate percentage homology or identity between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol. Lett. (1999) 174: 247-50; FEMS Microbiol. Lett. (1999) 177: 187-8).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percentage homology, preferably percentage sequence identity. The software typically does this as part of the sequence comparison and generates a numerical result.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

Codon Optimisation

The polynucleotides used in the present invention may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

Kit

In one aspect, the present invention provides a kit comprising: (i) the medicament described herein (e.g. the vector); and (ii) a solution that does not comprise the medicament for use in at least partially detaching the retina of a mammalian subject to form a subretinal bleb. Preferably the solution of component (ii) is balanced salt solution (BSS) or a similar buffer solution matched to the pH and osmolality of the subretinal space.

The medicament and solution that does not comprise the medicament (i.e. components (i) and (ii)) may be provided in suitable containers.

The kit may also include instructions for use, for example instructions for the method of delivering a medicament of the present invention.

### EXAMPLES

### Example 1

Standard Operating Procedure for AAV.REP1 Vector Administration and Dosing for the Treatment of Choroideremia

Equipment

The AAV.REP1 vector is supplied in labelled polypropylene vials, each containing 100 μL of vector suspension at a concentration of 1×10¹² genome particles (gp) per mL in 0.001% pluronic acid surfactant, PF68. Each 0.10 mL vial therefore contains 1×10¹¹ gp of vector in total. The equipment set up described below has been tested to deliver a predictable quantity of vector to the subretinal space.

Vector Loading

A total of 0.30 mL of undiluted vector suspension is drawn from 3 vector vials into a 1 mL BD Luer-Lok syringe (BD reference 309628, Becton Dickinson UK Ltd, Oxford, UK) fitted with a 19 gauge (G) BD Microlance needle (BD reference 301750) (FIG. 1A).

A second 1 mL BD Luer-Lok syringe is used to inject the vector. This “injector syringe” has had its white plunger removed but the rubber bung reinserted to the 0.35 mL mark in preparation for vector loading (FIG. 1B). This set-up is necessary to attach the “injector syringe” to the viscous fluid injection system of the vitrectomy machine.

The vector suspension is transferred from the first “drawing-up syringe” to the “injector syringe” by inserting the 19 G needle through the open tip of the latter. Care is taken to inject slowly so as to avoid bubbles or contact between the needle tip and rubber bung during the loading process.

Once loaded with vector, the “injector syringe” is fitted with a DORC 41 G Teflon subretinal injection needle (Dutch Ophthalmic Research Center International BV, Zuidland, The Netherlands) (FIG. 1C). The 41 G Teflon needle allows self-sealing entry through the neurosensory retina into the subretinal space. Care must be taken not to damage the fine tip. The injector syringe is then held vertically and tapped gently to clear any bubbles from the suspension.

Vector Injection System

In order to connect the vector loaded “injector syringe” to the vitrectomy machine (e.g. Alcon Accurus or Constellation), modification of the standard viscous fluid injection system is required. A syringe lock from the Bausch & Lomb Viscous Fluid Pack (CX5710, Bausch & Lomb Inc, NY, USA) has been custom modified to fit an 8.0 mm rubber O-ring (FIG. 2). The modification has been carried out by the Scientific Engineering Workshop Department of the University College London Institute of Ophthalmology, London, UK. The modified syringe lock has been re-sterilised by Andersen Caledonia, Lanarkshire, UK.

The “injector syringe” plugs onto the modified syringe lock with the O-ring providing an air-tight seal. The syringe lock, in turn, connects via a standard Luer-Lok air-filter and Easy Connection Tubing (ECT003-00, ALCHIMIA Srl, Ponte S. Nicolò, Italy) to an Alcon Accurus or Constellation vitrectomy machine (FIG. 3). Versions of the Easy Connection Tubing are available from ALCHIMIA for connecting to other vitrectomy machines.

Once connected, the viscous fluid injection option on the vitrectomy machine is set to 12 pounds per square inch (PSI) and the foot-pedal pressed gently to advance the bung forwards under air pressure. This pushes the vector suspension slowly into the 41 G subretinal injection needle. The needle has a 0.12 mL dead space and is therefore fully primed when the bung reaches 0.18 mL, however there may be losses from transfer so the vector can be advanced further until fluid is seen emerging from the tip of the needle. The surplus vector emerging from the tip is collected into one of the now empty polypropylene vector vials for subsequent safe disposal. The vector suspension should not be primed beyond 0.10 mL as this will limit the amount of vector that could be injected into the subretinal space. The join between the syringe and 41 G needle is inspected carefully for the presence of any bubbles during the loading procedure. If bubbles are a concern, the fluid injector may be switched to aspiration so that air is drawn up the 41 G needle with the bung returning to the 0.35 mL mark. The syringe may then be held vertically and tapped again before repriming the system. It is perfectly acceptable to add 0.10 mL of balanced salt solution (BSS) to increase the volume of the vector suspension if necessary. It is recommended that each vitrectomy machine being used is pre-tested with a solution of saline loaded using the same steps as above to check the precise settings that result in a steady drip of fluid from the 1 mL injection syringe when connected.

Subretinal Vector Injection

A foveal retinal detachment is first created by slow subretinal injection of BSS through a DORC 41 G Teflon needle, starting away from the fovea. Once the target area has been detached, a fixed amount of 1×10¹¹ gp in 0.10 mL of vector is injected using the loaded “injector syringe” prepared above into the bleb through the same retinotomy. A 90 dioptre BIOM lens is preferred for optimum visualisation during injections. If the original retinotomy cannot be identified then another suitable entry point is chosen. Under no circumstance should this be less than 1 mm from the fovea and great care must be taken if the bleb is small because the relative stretch of the overlying retina will be proportionately greater. The bottle height (or infusion pressure on the Alcon Constellation machine) is set to 15-20 cm. The vector suspension is injected in successive gentle pulses of the foot-pedal. With each wave of injection, the retina will be seen to stretch and blanch slightly as vector suspension is forced into it by hydrostatic force. The operating assistant must watch the bung position and advise the surgeon when the 0.10 mL total injection volume is approaching. It is however perfectly acceptable to inject a lower volume (down to 0.05 mL) if there is deemed to be too much retinal stretch.

An indentation search is performed gently to avoid vortexing of fluid inside the eye which might induce a Venturi suction effect over the self-sealing retinotomy. Laser should be applied to any peripheral breaks after relief of vitreous traction. Gas should be avoided where possible as it may induce a macular fold or displace subretinal vector to non-therapeutic regions.

Retinal Tears or Entry Site Breaks

Peripheral tears should be treated with laser and careful vitrectomy around the retinal tear to relieve any traction. Air or gas should not be used as this would displace the vector inferiorly away from the macula when the trial participant returns to the upright position. In cases of giant retinal tear, treatment should be confined to laser (or cryotherapy) only where possible, bearing in mind that the scar tissue formed as a result of chronic retinal degeneration in choroideremia will create a firm adherence that would limit progression of even large retinal tears compared with the norm.

Macular Hole During Retinal Detachment Phase

As a result of retinal degeneration in these patients, the macula is expected to be thinner than normal in choroideremia patients. This may put patients at an increased risk of macular hole formation during subretinal injections. In cases where a hole forms during the initial detachment phase, it should be closed by draining to air and closing the eye without administering the vector. The patient can be brought back for vector administration at a later date, once the retina has healed. Gas in the eye lasts considerably longer in choroideremia patients, therefore air or low concentration sulphur hexafluoride (10% SF₆) should be used (Zingernagel et al. (2013) Ophthalmology 120: 1592-6).

Macular Hole During Vector Injection Phase

If a macular hole occurs during vector injection, the vector will likely escape into the vitreous cavity, but most will be flushed out of the eye during fluid-air exchange, which will reduce the risk of inflammation. Where possible, the surgeon should get as much vector suspension into the subretinal space as possible and then wait 20-30 minutes for absorption of vector from the subretinal space into the retinal pigment epithelium, before draining to air over the optic disc. The patient should be recovered in the supine position and kept supine for 6-8 hours after surgery to allow maximal vector absorption from the subretinal space. Following that, the face down position can be adopted for a further 24 hours to flatten the retina and close the hole. Avoiding immediate face-down positioning gives more time for the vector suspension to be absorbed and the supine position limits movement of the eye which might otherwise vortex vector suspension out from the subretinal space into the vitreous cavity.

### Example 2

Preclinical Testing of Vector Dose

In order to test the vector dose emerging from the DORC 41 G cannula and 1 mL syringe, the injection equipment was tested at the Nationwide Children's Hospital Vector Core in Columbus, Ohio. A test batch of AAV.REP1 in 0.001% PF68 surfactant was made up as reported in MacLaren, R. E. et al. (2014) Lancet 383: 1129-37. The PCR and other test equipment used in this assay are maintained to GMP standards at the NCH Vector Core. The assays were performed following detailed discussions between the tester and the Chief Investigator of the clinical trial in relation to the precise steps and timings that would be involved in the operating theatre.

The titre of DNAse resistant particles (DRP/mL) was assessed by quantitative polymerase chain reaction (qPCR) on the vector at baseline, after injection through the Dorc 41 G cannula and on the vector remaining in the syringe. Two samples were tested from each group and at two separate dilutions, with forward and reverse primers directed against the CAG promoter sequence. The doubling cycle threshold (CT) was generated for each sample and the results showed no significant change in vector titre (10¹¹ gp/mL) after passage through the injection system (Table 1).

TABLE 1

| Sample | | | | | | Titer | Average |
|---|---|---|---|---|---|---|---|
| No. | Description | Dilution | Detector | CT | Quantity | (DRP/mL) | Titer |
| 1 | Baseline titre | 1000 | CAG | 26.5178 | 3.49E+05 | 1.397E+11 | 1.38E+11 |
| 1 | Baseline titre | 1000 | CAG | 26.8537 | 2.78E+05 | 1.112E+11 | |
| 2 | Baseline titre | 10000 | CAG | 29.6107 | 4.28E+04 | 1.713E+11 | |
| 2 | Baseline titre | 10000 | CAG | 30.0374 | 3.21E+04 | 1.283E+11 | |
| 3 | Vector injected | 1000 | CAG | 25.7056 | 6.06E+05 | 2.424E+11 | 1.87E+11 |
| 3 | Vector injected | 1000 | CAG | 26.3771 | 3.84E+05 | 1.537E+11 | |
| 4 | Vector injected | 10000 | CAG | 29.5299 | 4.52E+04 | 1.810E+11 | |
| 4 | Vector injected | 10000 | CAG | 29.5991 | 4.32E+04 | 1.727E+11 | |
| 5 | Vector in syringe | 1000 | CAG | 25.9066 | 5.29E+05 | 2.115E+11 | 2.13E+11 |
| 5 | Vector in syringe | 1000 | CAG | 25.8216 | 5.60E+05 | 2.241E+11 | |
| 6 | Vector in syringe | 10000 | CAG | 29.2757 | 5.38E+04 | 2.151E+11 | |
| 6 | Vector in syringe | 10000 | CAG | 29.3822 | 5.00E+04 | 2.001E+11 | |

### Example 3

Retinal Gene Therapy in Patients with Choroideremia

Patients and Study Design

Six male patients with a clinical phenotype of choroideremia and predicted null mutations in the CHM gene were enrolled into this multicentre trial after they provided written informed consent. The ages of patients 1-6 were 63 years, 47 years, 35 years, 55 years, 41 years, and 56 years, respectively. The patients represented different stages of the disease against which to assess the efficacy of the intervention—a normal foveal structure, partial foveal collapse, and complete foveal loss. The Gene Therapy Advisory Committee (UK Department of Health) provided ethics approval.

Vector Production

The AAV2 expression cassette comprised a chicken β actin promoter, established for long-term transduction of the retinal pigment epithelium in previous clinical trials of human retinal gene therapy (Jacobson, S. G. et al. (2012) Arch. Ophthalmol. 130: 9-24; Maguire, A. M. et al. (2008) N. Engl. J. Med. 358: 2240-48) and a Woodchuck hepatitis virus post-translational regulatory element (WPRE) downstream of the CHM cDNA (encoding REP1). WPRE is known to enhance AAV-mediated transgene expression (Loeb, J. E. et al. (1999) Hum. Gene Ther. 10: 2295-305) and was approved by the US Food and Drug Administration for a clinical trial of AAV2 for Parkinson's disease (LeWitt, P. A. et al. (2011) Lancet Neurol. 10: 309-19). This human REP1 sequence was previously shown in vitro to restore prenylation activity when delivered by an adenoviral vector to cells isolated from patients with choroideremia (Anand, V. et al. (2003) Vision Res. 43: 919-26). The subsequent preclinical data for the effects of this vector (AAV2.REP1) in restoring prenylation activity to human choroideremia fibroblasts and the electroretinogram in mouse choroideremia are described elsewhere (Tolmachova, T. et al. (2013) J. Mol. Med. (Berl) 91: 825-37.). PF68 surfactant was added to prevent non-specific binding of AAV particles to plastics inside the injection system (Bennicelli, J. et al. (2008) Mol. Ther. 16: 458-65).

Surgery

Because of the unpredictability of detachment of the retina in choroideremia, patients were injected with AAV2.REP1 as a two-step procedure. Surgery was first undertaken to detach the retina through a 41 G Teflon cannula (DORC International BV, Zuidland, Netherlands) using balanced salt solution (Alcon Laboratories, Fort Worth, Tex., USA). Once the retinal target area had been detached from the underlying retinal pigment epithelium, a fixed volume (0.1 mL) containing 1×10¹⁰ genome particles of AAV2.REP1 was injected through a fresh syringe into the subretinal space that had been created in the first five patients. In patient 6, a reduced dose of up to 6×10⁹ genome particles was injected. The vector was injected slowly through the same retinotomy, causing the detachment to extend further.

Clinical Assessments

All patients had best corrected visual acuity and contrast sensitivity measured in each eye separately after a standardised refraction.

Findings

Despite undergoing retinal detachment, which normally reduces vision, two patients with advanced choroideremia who had low baseline best corrected visual acuity gained 21 letters and 11 letters (more than two and four lines of vision). Four other patients with near normal best corrected visual acuity at baseline recovered to within one to three letters. Mean gain in visual acuity overall was 3·8 letters (SE 4·1). Maximal sensitivity measured with dark-adapted microperimetry increased in the treated eyes from 23·0 dB (SE 1·1) at baseline to 25·3 dB (1·3) after treatment (increase 2·3 dB [95% Cl 0·8-3·8]). In all patients, over the 6 months, the increase in retinal sensitivity in the treated eyes (mean 1·7 [SE 1·0]) was correlated with the vector dose administered per mm² of surviving retina (r=0·82, p=0·04). By contrast, small non-significant reductions (p>0·05) were noted in the control eyes in both maximal sensitivity (−0·8 dB [1·5]) and mean sensitivity (−1·6 dB [0·9]). One patient in whom the vector was not administered to the fovea re-established variable eccentric fixation that included the ectopic island of surviving retinal pigment epithelium that had been exposed to vector.

The initial results of this retinal gene therapy trial are consistent with improved rod and cone function that overcome any negative effects of retinal detachment.

All publications mentioned in the above specification are herein incorporated by reference. Various modifications and variations of the described methods of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention, which are obvious to those skilled in biochemistry, biotechnology, medicine or related fields, are intended to be within the scope of the following claims.

1. A method of using a vector comprising a transgene to treat a disease or condition of the eye, the method comprising the steps:
(a) administering a solution to a mammalian subject by subretinal injection in an amount effective to at least partially detach the retina to form a subretinal bleb, wherein the solution does not comprise the vector; and
(b) administering a medicament composition by subretinal injection into the bleb formed by step (a), wherein the medicament comprises the vector and is injected in an amount effective to treat the disease or condition;
wherein the transgene is expressible in cells of the mammalian subject.

2. The method of claim 1, wherein the vector is a viral vector.

3. The method of claim 2, wherein the viral vector is an AAV, retroviral, lentiviral or adenoviral vector.

4. The method of claim 1, wherein the transgene comprises a Rab escort protein-1 (REP1) or retinal pigment epithelium-specific 65 kDa protein (RPE65) open reading frame operably linked to an expression control sequence to promote expression in cells of the eye of the mammalian subject.

5. The method of claim 1, wherein the vector is delivered to cells of the neurosensory retina, retinal pigment epithelium and/or choroid.

6. The method of claim 1, wherein the medicament composition is administered by subretinal injection through the same retinotomy used to administer the solution.

7. The method of claim 1, wherein the subretinal injections are carried out using a subretinal injection needle to create self-sealing entry points in the neurosensory retina.

8. The method of claim 1, wherein the area of the retina to be injected is dyed with a blue vital dye before the subretinal injection of step (a) is carried out.

9. The method of claim 1, wherein the subretinal injections are at positions greater than or equal to about 1 mm from the fovea.

10. The method of claim 1, wherein the subretinal injections of steps (a) and/or (b) are made in a series of successive pulses to stretch and blanch the retina.

11. The method of claim 1, wherein the solution injected in step (a) is balanced salt solution (BSS).

12. The method of claim 1, wherein the disease or condition is a retinal dystrophy.

13. The method of claim 1, wherein the disease or condition is choroideremia, Leber congenital amaurosis, cone-rod dystrophy, macular dystrophy, cone dystrophy, achromatopsia, retinitis pigmentosa or age-related macular degeneration.

14. A method of treatment of a disease or condition of the eye, the method comprising administering a vector comprising a transgene by the steps:
(a) administering a solution to a mammalian subject by subretinal injection in an amount effective to at least partially detach the retina to form a subretinal bleb, wherein the solution does not comprise the vector; and
(b) administering a medicament composition by subretinal injection into the bleb formed by step (a), wherein the medicament comprises the vector and is injected in an amount effective to treat the disease or condition;
wherein the transgene is expressible in cells of the mammalian subject.

15. The method of claim 14, wherein the vector is a viral vector.

16. The method of claim 15, wherein the viral vector is an AAV, retroviral, lentiviral or adenoviral vector.

17. The method of claim 14, wherein the transgene comprises a Rab escort protein-1 (REP1) or retinal pigment epithelium-specific 65 kDa protein (RPE65) open reading frame operably linked to an expression control sequence to promote expression in cells of the eye of the mammalian subject.

18. The method of claim 14, wherein the vector is delivered to cells of the neurosensory retina, retinal pigment epithelium and/or choroid.

19. The method of claim 14, wherein the medicament composition is administered by subretinal injection through the same retinotomy used to administer the solution.

20. The method of claim 14, wherein the subretinal injections are carried out using a subretinal injection needle to create self-sealing entry points in the neurosensory retina.

21. The method of claim 14, wherein the area of the retina to be injected is dyed with a blue vital dye before the subretinal injection of step (a) is carried out.

22. The method of claim 14, wherein the subretinal injections are at positions greater than or equal to about 1 mm from the fovea.

23. The method of claim 14, wherein the subretinal injections of steps (a) and/or (b) are made in a series of successive pulses to stretch and blanch the retina.

24. The method of claim 14, wherein the solution injected in step (a) is balanced salt solution (BSS).

25. The method of claim 14, wherein the disease or condition is a retinal dystrophy.

26. The method of claim 14, wherein the disease or condition is choroideremia, Leber congenital amaurosis, cone-rod dystrophy, macular dystrophy, cone dystrophy, achromatopsia, retinitis pigmentosa or age-related macular degeneration.

27. A kit comprising: (i) the vector as defined in claim 1; and (ii) a solution that does not comprise the vector for use in at least partially detaching the retina of a mammalian subject to form a subretinal bleb.

28. The method of claim 1, wherein the vector is an AAV vector and the transgene comprises a Rab escort protein-1 (REP1) open reading frame operably linked to an expression control sequence to promote expression in cells of the eye of the mammalian subject.

29. The method of claim 14, wherein the vector is an AAV vector and the transgene comprises a Rab escort protein-1 (REP1) open reading frame operably linked to an expression control sequence to promote expression in cells of the eye of the mammalian subject.


SEQUENCE LISTING (7 sequences)

SEQ ID NO: 1 - protein, 653 aa, Homo sapiens
MADTLPSEFDVIVIGTGLPESIIAAACSRSGRRVLHVDSRSYYGGNWASFSFSGLLSWLKEYQENSDIVSDSPVWQDQILENEEAIALSRKDKTIQHVEVFCYASQDLHEDVEEAGALQKNHALVTSANSTEAADSAFLPTEDESLSTMSCEMLTEQTPSSDPENALEVNGAEVTGEKENHCDDKTCVPSTSAEDMSENVPIAEDTTEQPKKNRITYSQIIKEGRRFNIDLVSKLLYSRGLLIDLLIKSNVSRYAEFKNITRILAFREGRVEQVPCSRADVFNSKQLTMVEKRMLMKFLTFCMEYEKYPDEYKGYEEITFYEYLKTQKLTPNLQYIVMHSIAMTSETASSTIDGLKATKNFLHCLGRYGNTPFLFPLYGQGELPQCFCRMCAVFGGIYCLRHSVQCLVVDKESRKCKAIIDQFGQRIISEHFLVEDSYFPENMCSRVQYRQISRAVLITDRSVLKTDSDQQISILTVPAEEPGTFAVRVIELCSSTMTCMKGTYLVHLTCTSSKTAREDLESVVQKLFVPYTEMEIENEQVEKPRILWALYFNMRDSSDISRSCYNDLPSNVYVCSGPDCGLGNDNAVKQAETLFQEICPNEDFCPPPPNPEDIILDGDSLQPEASESSAIPEANSETFKESTNLGNLEESSE

SEQ ID NO: 2 - DNA, 1962 bp, Homo sapiens
atggcggatactctcccttcggagtttgatgtgatcgtaatagggacgggtttgcctgaatccatcattgcagctgcatgttcaagaagtggccggagagttctgcatgttgattcaagaagctactatggaggaaactgggccagttttagcttttcaggactattgtcctggctaaaggaataccaggaaaacagtgacattgtaagtgacagtccagtgtggcaagaccagatccttgaaaatgaagaagccattgctcttagcaggaaggacaaaactattcaacatgtggaagtattttgttatgccagtcaggatttgcatgaagatgtcgaagaagctggtgcactgcagaaaaatcatgctcttgtgacatctgcaaactccacagaagctgcagattctgccttcctgcctacggaggatgagtcattaagcactatgagctgtgaaatgctcacagaacaaactccaagcagcgatccagagaatgcgctagaagtaaatggtgctgaagtgacaggggaaaaagaaaaccattgtgatgataaaacttgtgtgccatcaacttcagcagaagacatgagtgaaaatgtgcctatagcagaagataccacagagcaaccaaagaaaaacagaattacttactcacaaattattaaagaaggcaggagatttaatattgatttagtatcaaagctgctgtattctcgaggattactaattgatcttctaatcaaatctaatgttagtcgatatgcagagtttaaaaatattaccaggattcttgcatttcgagaaggacgagtggaacaggttccgtgttccagagcagatgtctttaatagcaaacaacttactatggtagaaaagcgaatgctaatgaaatttcttacattttgtatggaatatgagaaatatcctgatgaatataaaggatatgaagagatcacattttatgaatatttaaagactcaaaaattaacccccaacctccaatatattgtcatgcattcaattgcaatgacatcagagacagccagcagcaccatagatggtctcaaagctaccaaaaactttcttcactgtcttgggcggtatggcaacactccatttttgtttcctttatatggccaaggagaactcccccagtgtttctgcaggatgtgtgctgtgtttggtggaatttattgtcttcgccattcagtacagtgccttgtagtggacaaagaatccagaaaatgtaaagcaattatagatcagtttggtcagagaataatctctgagcatttcctcgtggaggacagttactttcctgagaacatgtgctcacgtgtgcaatacaggcagatctccagggcagtgctgattacagatagatctgtcctaaaaacagattcagatcaacagatttccattttgacagtgccagcagaggaaccaggaacttttgctgttcgggtcattgagttatgttcttcaacgatgacatgcatgaaaggcacctatttggttcatttgacttgcacatcttctaaaacagcaagagaagatttagaatcagttgtgcagaaattgtttgttccatatactgaaatggagatagaaaatgaacaagtagaaaagccaagaattctgtgggctctttacttcaatatgagagattcgtcagacatcagcaggagctgttataatgatttaccatccaacgtttatgtctgctctggcccagattgtggtttaggaaatgataatgcagtcaaacaggctgaaacacttttccaggaaatctgccccaatgaagatttctgtccccctccaccaaatcctgaagacattatccttgatggagacagtttacagccagaggcttcagaatccagtgccataccagaggctaactcggagactttcaaggaaagcacaaaccttggaaacctagaggagtcctctgaataa

SEQ ID NO: 3 - DNA, 1992 bp, Homo sapiens
gatatcgaattcctgcagcccggcggcaccatggcggatactctcccttcggagtttgatgtgatcgtaatagggacgggtttgcctgaatccatcattgcagctgcatgttcaagaagtggccggagagttctgcatgttgattcaagaagctactatggaggaaactgggccagttttagcttttcaggactattgtcctggctaaaggaataccaggaaaacagtgacattgtaagtgacagtccagtgtggcaagaccagatccttgaaaatgaagaagccattgctcttagcaggaaggacaaaactattcaacatgtggaagtattttgttatgccagtcaggatttgcatgaagatgtcgaagaagctggtgcactgcagaaaaatcatgctcttgtgacatctgcaaactccacagaagctgcagattctgccttcctgcctacggaggatgagtcattaagcactatgagctgtgaaatgctcacagaacaaactccaagcagcgatccagagaatgcgctagaagtaaatggtgctgaagtgacaggggaaaaagaaaaccattgtgatgataaaacttgtgtgccatcaacttcagcagaagacatgagtgaaaatgtgcctatagcagaagataccacagagcaaccaaagaaaaacagaattacttactcacaaattattaaagaaggcaggagatttaatattgatttagtatcaaagctgctgtattctcgaggattactaattgatcttctaatcaaatctaatgttagtcgatatgcagagtttaaaaatattaccaggattcttgcatttcgagaaggacgagtggaacaggttccgtgttccagagcagatgtctttaatagcaaacaacttactatggtagaaaagcgaatgctaatgaaatttcttacattttgtatggaatatgagaaatatcctgatgaatataaaggatatgaagagatcacattttatgaatatttaaagactcaaaaattaacccccaacctccaatatattgtcatgcattcaattgcaatgacatcagagacagccagcagcaccatagatggtctcaaagctaccaaaaactttcttcactgtcttgggcggtatggcaacactccatttttgtttcctttatatggccaaggagaactcccccagtgtttctgcaggatgtgtgctgtgtttggtggaatttattgtcttcgccattcagtacagtgccttgtagtggacaaagaatccagaaaatgtaaagcaattatagatcagtttggtcagagaataatctctgagcatttcctcgtggaggacagttactttcctgagaacatgtgctcacgtgtgcaatacaggcagatctccagggcagtgctgattacagatagatctgtcctaaaaacagattcagatcaacagatttccattttgacagtgccagcagaggaaccaggaacttttgctgttcgggtcattgagttatgttcttcaacgatgacatgcatgaaaggcacctatttggttcatttgacttgcacatcttctaaaacagcaagagaagatttagaatcagttgtgcagaaattgtttgttccatatactgaaatggagatagaaaatgaacaagtagaaaagccaagaattctgtgggctctttacttcaatatgagagattcgtcagacatcagcaggagctgttataatgatttaccatccaacgtttatgtctgctctggcccagattgtggtttaggaaatgataatgcagtcaaacaggctgaaacacttttccaggaaatctgccccaatgaagatttctgtccccctccaccaaatcctgaagacattatccttgatggagacagtttacagccagaggcttcagaatccagtgccataccagaggctaactcggagactttcaaggaaagcacaaaccttggaaacctagaggagtcctctgaataa

SEQ ID NO: 4 - protein, 533 aa, Homo sapiens
MSIQVEHPAGGYKKLFETVEELSSPLTAHVTGRIPLWLTGSLLRCGPGLFEVGSEPFYHLFDGQALLHKFDFKEGHVTYHRRFIRTDAYVRAMTEKRIVITEFGTCAFPDPCKNIFSRFFSYFRGVEVTDNALVNVYPVGEDYYACTETNFITKINPETLETIKQVDLCNYVSVNGATAHPHIENDGTVYNIGNCFGKNFSIAYNIVKIPPLQADKEDPISKSEIVVQFPCSDRFKPSYVHSFGLTPNYIVFVETPVKINLFKFLSSWSLWGANYMDCFESNETMGVWLHIADKKRKKYLNNKYRTSPFNLFHHINTYEDNGFLIVDLCCWKGFEFVYNYLYLANLRENWEEVKKNARKAPQPEVRRYVLPLNIDKADTGKNLVTLPNTTATAILCSDETIWLEPEVLFSGPRQAFEFPQINYQKYCGKPYTYAYGLGLNHFVPDRLCKLNVKTKETWVWQEPDSYPSEPIFVSHPDALEEDDGVVLSVVVSPGAGQKPAYLLILNAKDLSEVARAEVEINIPVTFHGLFKKS

SEQ ID NO: 5 - DNA, 934 bp, Homo sapiens
attgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 6 - DNA, 270 bp, Bos taurus
tcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgggg

SEQ ID NO: 7 - DNA, 588 bp, Marmota monax
atcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc